(19) United States
(12) Patent Application Publication
(10) Pub. No.: US 2025/0001060 A1
(43) Pub. Date: Jan. 2, 2025

(54) PERITONEAL DIALYSIS SYSTEM HAVING A PATIENT LINE FILTER

(71) Applicants: BAXTER INTERNATIONAL INC., Deerfield, IL (US); BAXTER HEALTHCARE SA, Glattpark (Opfikon) (CH)

(72) Inventors: Serhan ACIKGOZ, Deerfield, IL (US); Christof BECK, Deerfield, IL (US); Rainer BLICKLE, Deerfield, IL (US); Reinhold BUCK, Deerfield, IL (US); Bjorn ERICSON, Lund (CH); Oskar Erik Frode STYRBJORN FALLMAN, Lund (CH); Ralf FLIEG, Deerfield, IL (US); Torsten KNOER, Deerfield, IL (US); Bernd KRAUSE, Deerfield, IL (US); John Sterling NORMAN, Deerfield, IL (US); Michael Patrick MORRISSEY, Deerfield, IL (US); Steffen WAGNER, Deerfield, IL (US)

(21) Appl. No.: 18/709,614

(22) PCT Filed: Nov. 18, 2022
(86) PCT No.: PCT/US22/80119

§ 371 Date: May 13, 2024

Related U.S. Application Data
(60) Provisional application No. 63/291,058, filed on Dec. 17, 2021.

(51) Int. Cl. A61M 1/28; A61M 1/14

(52) U.S. Cl. CPC A61M 1/288; A61M 1/154; A61M 1/1563; A61M 1/159; A61M 1/285; A61M 2205/3344; A61M 2205/7518; A61M 2205/7536

(57) ABSTRACT

A peritoneal dialysis (“PD”) system (10) includes a PD machine (20); a patient line (50) extending from the PD machine (20); and a filter set (100) in fluid communication with the patient line (50), the filter set (100) including at least one filter membrane (120a, 120b, such as a bacteria reduction or sterilizing grade filter membrane) positioned and arranged such that fresh PD fluid flows through the at least one filter membrane (120a, 120b) prior to exiting the filter set (100), the filter set (100) further including a used PD fluid tube (106u) positioned and arranged to carry used PD fluid past the at least one filter membrane (120a, 120b) without contacting the at least one filter membrane (120a, 120b). A method for priming filter set (100) is also disclosed.

### PRIORITY CLAIM

The present application claims priority to and the benefit of U.S. Provisional Application No. 63/291,058, filed on Dec. 17, 2021, the entire contents of which are hereby incorporated by reference.

### BACKGROUND

The present disclosure relates generally to medical fluid treatments and in particular to the filtering of treatment fluid during dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

PD fluid needs to be sterile or very near sterile because it is injected into the patient's peritoneal cavity, and is accordingly considered a drug. While bagged PD fluid is typically properly sterilized for treatment, PD fluid made online or PD machines or cyclers that employ disinfection may need additional sterilization.

There is accordingly a need for an effective, low cost way of providing additional sterilization to fresh PD fluid before it is delivered to a patient.

### SUMMARY

The present disclosure provides a peritoneal dialysis (“PD”) system having a PD machine or cycler that pumps fresh PD fluid through a patient line to a patient and removes used PD fluid from the patient via the patient line. The patient line may be reusable or disposable and in either case operates with and fluidly communicates with a filter set. If the patient line is reusable, the reusable patient line is connected to the filter set at the time of treatment. If the patient line is disposable, the filter set is merged into the disposable patient line in one embodiment. In either configuration a distal end of the filter set may be connected to the patient's transfer set, which in turn communicates fluidly with the patient's indwelling catheter.

The PD machine or cycler may include a durable PD fluid pump that pumps PD fluid through the pump itself without using a disposable component, or a disposable type PD fluid pump including a pump actuator that actuates a disposable, fluid-contacting pumping component, such as a peristaltic pump tube or a flexible pumping chamber. The PD machine or cycler also includes a plurality of valves, which may likewise be flow-through and durable without operating with a disposable component, or be disposable type valves having valve actuators that actuate a disposable, fluid-contacting valve component, such as a tube segment or a cassette-based valve seat.

The pumps and valves are under the automatic control of a control unit provided by the machine or cycler. In an embodiment, the valves include a fresh PD fluid valve that the control unit opens to allow the PD fluid pump to pump fresh PD fluid through a fresh PD fluid lumen of a dual lumen patient line to the patient. The valves also include a used PD fluid valve that the control unit opens to allow the PD fluid pump to pump used PD fluid from the patient through a used PD fluid lumen of the dual lumen patient line. It should be appreciated that while a single PD fluid pump may be used, dedicated fresh and used PD fluid pumps may be used alternatively. Also, a single PD fluid pump may include multiple pumping chambers for more continuous PD fluid flow.

The fresh and used PD fluid lumens may again be reusable or disposable. In the instance in which the fresh and used PD fluid lumens are reusable, the lumens terminate with a connector that connects to a lumen-side connector of the filter set, which may be sealed to (e.g., ultrasonically sealed, heat sealed, solvent bonded, laser welded or sealed via ultraviolet (“UV”) cured adhesive) or molded with a body of the filter set. The body is in turn sealed to (e.g., ultrasonically sealed, heat sealed, solvent bonded, laser welded or sealed via UV cured adhesive) or molded with a transfer set-side connector that either connects directly to a mating connector of the patient's transfer set or to a mating connector of a short tube placed between the body and the patient's transfer set. The transfer set-side connector may alternatively be placed at the end of a short tube that extends form the body. Here, the body provides (e.g., is molded with) a transfer set-side port to which the short tube extends into or over for welding to the port. The body, lumen-side connector, and transfer set-side connector or transfer set-side port may be referred to herein as a filter housing.

The lumen-side connector and the body form a fresh PD fluid passageway and a used PD fluid passageway. The fresh PD fluid passageway extends through a fresh PD fluid port in the lumen-side connector and towards a wall located within the body of the filter housing. The wall forces the fresh PD fluid to split outwardly in two directions forming a first outer compartment fresh PD fluid pathway and a second outer compartment fresh PD fluid pathway. The first outer compartment fresh PD fluid pathway extends in a first direction along and then past the wall and makes a turn, such as a right angle turn, into a first outer compartment, which resides over the outside of a first flat sheet filter membrane. The second outer compartment fresh PD fluid pathway extends in a second direction along and then past the wall and likewise makes a turn, such as a right angle turn, into a second outer compartment, which resides over the outside of a second flat sheet filter membrane. The fresh PD fluid is pressurized within the first and second outer compartments. The pressurization forces the fresh PD fluid through the filter membranes and into an inner compartment of the body, which is bounded in part by the inner surfaces of the first and second flat sheet filter membrane.

The filter membranes may be bacteria reduction or sterilizing grade hydrophilic membranes, which may be formed with porous walls having a pore size of about 0.2 micron, through which the fresh PD fluid flows for further filtration. Providing multiple flat sheet membranes enables the membranes and thus the filter housing of the filter set to be shorter, while providing the necessary filtration surface area needed over multiple patient fills of a PD treatment. A shorter filter housing is better for patient comfort because the patient is typically sleeping near the filter set during treatment.

Fresh and further filtered PD fluid flows in one embodiment from the inner compartment of the body, through the transfer set-side port, through the short tube of the filter set, and through the patient's transfer set, into the patient's peritoneal cavity. The transfer set-side port in one embodiment includes an internal stop that sets an inserted location of the short tube, e.g., flexible tube, or at least provides a location past which the short tube can no longer be inserted into the transfer set-side port. The internal stop is in one embodiment set off from the end of an internal used PD fluid tube by a desired distance. The used PD fluid tube may be molded with a primary portion of the body. The used PD fluid tube extends through the inner compartment of the body to a used PD fluid port provided by the lumen-side connector. The used PD fluid port is in sealed fluid communication during operation to the used PD fluid lumen of the dual lumen patient line.

The used PD fluid tube enables used PD fluid to be pulled through the body of the filter housing without contacting and potentially clogging either of the filter membranes. The used PD fluid tube also provides a clear, straight path for the used PD fluid, which helps to mitigate against pressure losses due to the filter set. While it is fluidically possible for used PD fluid to flow along the outside of the used PD fluid tube into the inner compartment of the body, negative pressure is applied only from within the used PD fluid tube, so there is little incentive for used PD fluid flow along the outside of the used PD fluid tube. Likewise, while it is fluidically possible for fresh PD fluid to flow in reverse up the used PD fluid tube, the change in direction required makes such a path much more tortuous than simply flowing through the transfer set-side port to the patient. Also, the used PD fluid tube and the used PD fluid lumen of the dual lumen patient tube are likely full of PD fluid during a patient fill, and the used PD fluid lumen is closed off at the PD machine or cycler, so there is little or no room for fresh PD fluid to enter the used PD fluid tube.

The inner compartment of the body is provided in one embodiment with a series of ribs that support the first and second flat sheet filter membranes when placed under negative fluid pressure. In this manner, the filter membranes and the inner compartment may be as large as they need to be to provide a desired filtration capacity. The series of ribs and their supporting structure are co-molded with the used PD fluid tube and the sidewalls of the body in one embodiment. The first and second flat sheet filter membranes are sealed in place to inner portions of the sidewalls of the body via ultrasonic sealing, heat sealing, solvent bonding, laser welding or sealing via UV cured adhesive.

The body of the filter housing includes first and second lids, which may be formed from the same material as the remainder of the body of the filter housing. The first and second lids may be ultrasonically sealed, heat sealed, solvent bonded, laser welded or sealed via UV cured adhesive to outer portions of the sidewalls of the body. A tongue and groove fit may be provided between the first and second lids and the sidewalls to center the lids for sealing and/or to serve as a flash trap for material melted by the weld or other process used to seal the lids to the body. The first and second lids respectively form the outsides of the first and second outer compartments into which fresh PD fluid flows before passing through the filter membranes.

The first and second lids may be formed with one or more air vent. Each vent is covered on the inside of the lid with a hydrophobic membrane, which may be ultrasonically sealed, heat sealed, solvent bonded, laser welded or sealed via UV cured adhesive to the inside surface of the lid and around the at least one vent opening. The one or more hydrophobic membrane allows air to be vented to atmosphere as the fresh PD fluid is pressurized within the outer compartments of the body prior to being filtered through the hydrophilic membranes, which may improve the performance of the membranes in addition to removing air from the filter set.

A gasket, such as a silicone or polyvinyl chloride (“PVC”) rubber gasket, is fitted onto and/or into the fresh and used PD fluid ports of the lumen-side connector of the filter housing. The gasket provides a double seal against the mating patient line connector in one embodiment. The patient line connector includes fresh and used ports that extend into the fresh and used PD fluid ports of the lumen-side connector. The gasket provides port seals between the mated fresh and used PD fluid ports of the patient line connector and the lumen-side connector. The port seals and the fresh and used ports of the patient line connector may be tapered so as to narrow while extending into the filter housing. The port seals are first seals. The patient line connector includes a flange having a raised continuous rib that extends around the flange. The flange and the rib are translated towards a flange portion of the gasket when the patient line connector is connected to the lumen-side connector. When the patient line connector is fully connected to the lumen-side connector, the raised rib extends into the deformable flange portion of gasket, which compresses around the raised rib to form a second seal.

Used PD fluid removed through the patient's transfer set travels under negative pressure through the filter set via the used PD fluid tube (thus bypassing the filter membranes), through the used PD fluid lumen of the dual lumen patient line, and back to the machine or cycler. The machine or cycler pumps the used PD fluid under positive pressure to drain. The cycler includes a pressure sensor located along the used PD fluid side of its internal tubing, which measures the negative pressure applied by the PD fluid pump to the used PD fluid during a patient drain. That same pressure sensor may be used during a patient fill to measure the positive pumping pressure (which is transmitted back through the used PD fluid tube of the filter set and used PD fluid lumen of the patient line to the pressure sensor), which is desirable because the measured pressure is of the fresh PD fluid downstream (after filtration) of the filter membranes. The measured pressure accordingly takes into account any pressure drop across the filter membranes, which more accurately reflects the pressure at which the PD fluid is being delivered to the patient.

Also described herein are multiple methods for priming the filter set of the present disclosure. In a first embodiment, the filter set may be clipped at the cycler housing, wherein the short tube is initially not connected to the patient's transfer set. Here, a cap provided at the end of the short tube acts to force fresh PD fluid used for priming, and thus air, back up a used PD fluid side of the filter set towards drain. In a second embodiment, the filter set is not clipped at the cycler housing and the short tube is initially connected to the patient's transfer set. Here, a twist clamp provided with the patient's transfer set acts to force fresh PD fluid used for priming, and thus air, back up a used PD fluid side of the filter set. The control unit then causes fresh PD fluid to be primed through the fresh PD fluid lumen, the body of filter set, and a portion of the used PD fluid lumen via the PD fluid pump. Fresh and used PD fluid valves are sequenced to do so. Then, with all but the short tube primed, the patient is prompted in the first embodiment to connect the short tube to the patient's transfer set and to open the transfer set clamp. In the second embodiment (short tube already connected), the patient is prompted to open the transfer set clamp. In either case, the control unit then causes, e.g., as part of an initial drain, the PD fluid pump to apply negative pressure through the used PD fluid lumen, which pulls used PD fluid from the patient to prime the short tube.

In light of the disclosure set forth herein, and without limiting the disclosure in any way, in a first aspect of the present disclosure, which may be combined with any other aspect, or portion thereof, a peritoneal dialysis (“PD”) system includes a PD machine: a patient line extending from the PD machine; and a filter set in fluid communication with the patient line, the filter set including at least one filter membrane positioned and arranged such that fresh PD fluid flows through the at least one filter membrane prior to exiting the filter set, the filter set further including a used PD fluid tube positioned and arranged to carry used PD fluid past the at least one filter membrane without contacting the at least one filter membrane.

In a second aspect of the present disclosure, which may be combined with any other aspect, or portion thereof, the patient line is a dual lumen patient line including a fresh PD fluid lumen and a used PD fluid lumen, the used PD fluid lumen placed in fluid communication with the used PD fluid tube.

In a third aspect of the present disclosure, which may be combined with any other aspect, or portion thereof, the filter set includes a fresh PD fluid port for fluid communication with the fresh PD fluid lumen and a used PD fluid port for fluid communication with the used PD fluid lumen.

In a fourth aspect of the present disclosure, which may be combined with any other aspect, or portion thereof, the used PD fluid tube is in fluid communication with the used PD fluid port.

In a fifth aspect of the present disclosure, which may be combined with any other aspect, or portion thereof, the filter set includes a compressible gasket configured to seal the fresh and used PD fluid ports to a patient line connector located at an end of the dual lumen patient line.

In a sixth aspect of the present disclosure, which may be combined with any other aspect, or portion thereof, the filter set includes first and second filter membranes separated by an inner compartment into which fresh PD fluid is filtered by the first and second filter membranes, and wherein the used PD fluid tube extends through the inner compartment.

In a seventh aspect of the present disclosure, which may be combined with any other aspect, or portion thereof, the PD system includes at least one rib located within the inner compartment for supporting the first and second filter membranes.

In an eighth aspect of the present disclosure, which may be combined with any other aspect, or portion thereof, the filter set includes a transfer set-side port within which fresh and used PD fluid flows, the used PD fluid tube extending into the transfer set-side port.

In a ninth aspect of the present disclosure, which may be combined with any other aspect, or portion thereof, the transfer set-side port defines an internal stop against which a tube may be abutted, the internal stop spaced from an end of the used PD fluid tube extending into the transfer set-side port.

In a tenth aspect of the present disclosure, which may be combined with any other aspect, or portion thereof, the filter set includes at least one outer compartment sized to displace the fresh PD fluid across an upstream side of the at least one filter membrane.

In an eleventh aspect of the present disclosure, which may be combined with any other aspect, or portion thereof, the at least one filter membrane is a flat sheet filter membrane.

In a twelfth aspect of the present disclosure, which may be combined with any other aspect, or portion thereof, the filter set includes a deflecting wall positioned and arranged to displace incoming fresh PD fluid towards the at least one outer compartment.

In a thirteenth aspect of the present disclosure, which may be combined with any other aspect, or portion thereof, the filter set includes at least one lid cooperating with the at least one filter membrane to form the at least one outer compartment.

In a fourteenth aspect of the present disclosure, which may be combined with any other aspect, or portion thereof, the at least one lid includes at least one vent opening and at least one hydrophobic membrane sealingly covering the at least one vent opening.

In a fifteenth aspect of the present disclosure, which may be combined with any other aspect, or portion thereof, the filter set includes at least one sidewall, wherein the at least one lid is sealed to an outer portion of the at least one sidewall, and wherein the least one filter membrane is sealed to an inner portion of the at least one sidewall.

In a sixteenth aspect of the present disclosure, which may be combined with any other aspect, or portion thereof, the filter set is configured to connect directly to a patient's transfer set, or wherein the filter set includes a flexible tube configured to connect to the patient's transfer set.

In a seventeenth aspect of the present disclosure, which may be combined with any other aspect, or portion thereof, the PD machine includes a pressure sensor positioned and arranged to sense the pressure of fresh PD fluid downstream from the at least one filter membrane during a patient fill.

In an eighteenth aspect of the present disclosure, which may be combined with any other aspect, or portion thereof, the at least one filter membrane is a bacteria reduction filter membrane or a sterilizing grade filter membrane.

In a nineteenth aspect of the present disclosure, which may be combined with any other aspect, or portion thereof, a peritoneal dialysis (“PD”) system includes a PD machine; a patient line extending from the PD machine; and a filter set in fluid communication with the patient line, the filter set including first and second filter membranes separated by an inner compartment into which fresh PD fluid is filtered by the first and second filter membranes, and wherein the filter set is further configured such that used PD fluid flows through the inner compartment.

In a twentieth aspect of the present disclosure, which may be combined with any other aspect, or portion thereof, the PD system includes at least one rib located within the inner compartment for supporting the first and second filter membranes, the used PD fluid flowing around the at least one rib before exiting the inner compartment.

In a twenty-first aspect of the present disclosure, which may be combined with any other aspect, or portion thereof, a filter set includes a body; at least one filter membrane positioned and arranged within the body such that fresh peritoneal dialysis (“PD”) fluid flows through the at least one filter membrane prior to exiting the filter set; and a used PD fluid tube positioned and arranged within the body to carry used PD fluid past the at least one filter membrane without contacting the at least one filter membrane.

In a twenty-second aspect of the present disclosure, which may be combined with any other aspect, or portion thereof, a filter set includes a body: at least one filter membrane positioned and arranged within the body such that fresh peritoneal dialysis (“PD”) fluid flows through the at least one filter membrane prior to exiting the filter set; and at least one lid including at least one vent opening and at least one protective rib located adjacent to the at least one vent opening to maintain the at least one vent opening in an uncovered condition during operation.

In a twenty-third aspect of the present disclosure, which may be combined with any other aspect, or portion thereof, a method is provided for priming a filter set connected to a dual lumen patient line, wherein during treatment a tube is located between the filter set and a patient's transfer set, the method including (i) delivering fresh peritoneal dialysis (“PD”) fluid through a fresh PD fluid lumen of the dual lumen patient line to the filter set: (ii) forcing the fresh PD fluid through at least one filter membrane of the filter set, so that the fresh PD fluid displaces air towards a used PD fluid lumen of the dual lumen patient line; and pulling used PD fluid from the patient, through the patient's transfer set, through the tube, through a used PD fluid portion of the filter set, and into the used PD fluid lumen of the dual lumen patient line.

In a twenty-fourth aspect of the present disclosure, which may be combined with any other aspect, or portion thereof, pulling used PD fluid is provided as part of an initial patient drain.

In a twenty-fifth aspect of the present disclosure, which may be combined with any other aspect, or portion thereof, wherein between forcing the fresh PD fluid through the at least one filter membrane and pulling used PD fluid from the patient, the patient is prompted to connect the tube to the patient's transfer set.

In a twenty-sixth aspect of the present disclosure, which may be combined with any other aspect, or portion thereof, wherein between forcing the fresh PD fluid through the at least one filter membrane and pulling used PD fluid from the patient, the patient is prompted to open a clamp of the patient's transfer set.

In a twenty-seventh aspect of the present disclosure, which may be combined with any other aspect, or portion thereof, air is primed through at least one vent opening of the filter set while delivering fresh PD fluid through the fresh PD fluid lumen of the dual lumen patient line.

In a twenty-eighth aspect of the present disclosure, which may be combined with any other aspect, or portion thereof, a fresh PD fluid valve is open and a used PD fluid valve is closed while delivering fresh PD fluid through the fresh PD fluid lumen of the dual lumen patient line.

In a twenty-ninth aspect of the present disclosure, which may be combined with any other aspect, or portion thereof, a used PD fluid valve is open while forcing the fresh PD fluid through the at least one filter membrane of the filter set.

In a thirtieth aspect of the present disclosure, which may be combined with any other aspect, or portion thereof, a used PD fluid valve is open while pulling used PD fluid from the patient.

In a thirty-first aspect of the present disclosure, which may be combined with any other aspect, or portion thereof, the method includes accumulating known volume pump strokes to control a volume pumped to force the fresh PD fluid through the at least one filter membrane.

In a thirty-second aspect of the present disclosure, which may be combined with any other aspect, or portion thereof, the method includes sensing a pressure increase to transition from (i) delivering fresh PD fluid through the fresh PD fluid lumen of the dual lumen patient line to the filter set to (ii) forcing the fresh PD fluid through the at least one filter membrane.

In a thirty-third aspect of the present disclosure, which may be combined with any other aspect, or portion thereof, the used PD fluid portion of the filter set includes a used PD fluid tube.

In a thirty-fourth aspect of the present disclosure, which may be combined with any other aspect, or portion thereof, the used PD fluid pulled from the patient is residual effluent from a previous treatment left for the purpose of priming the tube.

In a thirty-fifth aspect of the present disclosure, which may be combined with any other aspect, or portion thereof, a volume of the residual effluent is at least 50 ml.

In a thirty-sixth aspect of the present disclosure, which may be combined with any other aspect, or portion thereof, any of the features, functionality and alternatives described in connection with any one or more of FIGS. 1 to 8B may be combined with any of the features, functionality and alternatives described in connection with any other of FIGS. 1 to 8B.

In light of the above aspects and the description herein, it is accordingly an advantage of the present disclosure to provide a filter set that operates with a dual lumen patient line.

It is another advantage of the present disclosure to provide a filter set that filters fresh PD fluid and allows used PD fluid to pass with limited contact to or perhaps without contacting the filter membranes.

It is a further advantage of the present disclosure to provide a filter set that sizes and spaces the filter membranes efficiently to reduce size and aid patient comfort.

It is yet another advantage of the present disclosure to provide a filter set that vents air from the fresh PD fluid before it is further filtered by the filter set.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for peritoneal dialysis system having a filter set of the present disclosure.

FIG. 2 is an exploded perspective view of one embodiment for a filter set of the present disclosure.

FIG. 3 is a sectioned perspective view highlighting a fresh PD fluid inlet pathway of one embodiment for a filter set of the present disclosure.

FIG. 4 is a sectioned perspective view highlighting a used PD fluid outlet pathway of one embodiment for a filter set of the present disclosure.

FIG. 5 is a sectioned perspective view highlighting a used PD fluid tube and filter membrane support ribs of one embodiment for a filter set of the present disclosure.

FIG. 6 is a sectioned elevation view highlighting the outer fresh PD fluid compartments and hydrophobic membranes of one embodiment for a filter set of the present disclosure.

FIGS. 7A and 7B are sectioned perspective views of a second embodiment for a filter set of the present disclosure.

FIGS. 8A and 8B are perspective views illustrating one embodiment for the filter set body of the present disclosure having ribbed, vent opening lids.

### DETAILED DESCRIPTION

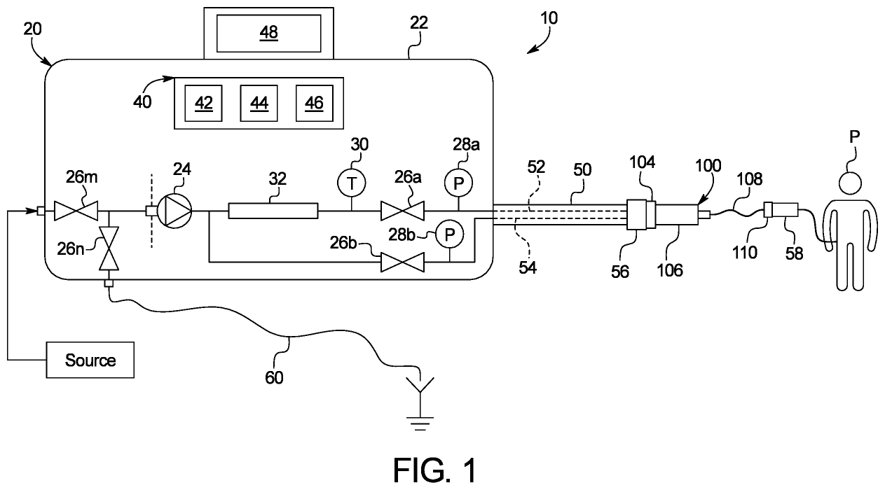

Referring now to the drawings and in particular to FIG. 1, a peritoneal dialysis (“PD”) system 10 is illustrated. PD system 10 includes a PD machine or cycler 20 that pumps fresh PD fluid through a patient line 50 to a patient P and removes used PD fluid from patient P via patient line 50. Patient line 50 may be reusable or disposable and in either case operates with and fluidly communicates with a filter set 100. If patient line 50 is reusable, the reusable patient line is connected to filter set 100 at the time of treatment. If patient line 50 is instead disposable, filter set 100 is merged into or formed with disposable patient line 50 in one embodiment. In either configuration, a distal end of filter set 100 may be connected to the patient's transfer set 58, which in turn communicates fluidly with the indwelling catheter of patient P.

PD machine or cycler 20 may include a housing 22 providing a durable PD fluid pump 24 that pumps PD fluid through the pump itself without using a disposable component. Examples of durable pumps that may be used for PD fluid pump 24 include piston pumps, gear pumps and centrifugal pumps. Certain durable pumps, such as piston pumps are inherently accurate, so that machine or cycler 20 does not require additional volumetric control components. Other durable pumps, such as gear pumps and centrifugal pumps may not be as accurate, such that machine or cycler 20 provides a volumetric control device such as one or more flowmeter (not illustrated).

Pump 24 may alternatively be a disposable type PD fluid pump, which includes a pump actuator that actuates a disposable, fluid-contacting pumping component, such as a peristaltic pump tube or a flexible pumping chamber. Examples of disposable PD fluid pumps that may be used for PD fluid pump 24 include rotary or linear peristaltic pump actuators that actuate tubing, pneumatic pump actuators that actuate cassette sheeting, electromechanical pump actuators that actuate cassette sheeting and platen pump actuators that actuate tubing. It should be appreciated that while a single PD fluid pump 24 may be used, dedicated fresh and used PD fluid pumps may be used alternatively. Also, single PD fluid pump 24 may include multiple pumping chambers for more continuous PD fluid flow.

PD machine or cycler 20 also includes a plurality of valves 26a, 26b, 26m, 26n which may likewise be flow-through and durable without operating with a disposable component, or be disposable type valves having valve actuators that actuate a disposable, fluid-contacting valve component, such as a tube segment or a cassette-based valve seat. Examples of durable valves that may be used for valves 26a, 26b, 26m, 26n include flow-through solenoid valves. Such valves may be two-way or three-way valves. Examples of disposable valves that may be used for valves 26a, 26b, 26m, 26n include solenoid pinch valves that pinch closed flexible tubing, pneumatic valve actuators that actuate cassette sheeting, and electromechanical valve actuators that actuate cassette sheeting.

Machine or cycler 20 likely includes many valves 26a to 26n. For ease of illustration, machine or cycler 20 is shown having a fresh PD fluid valve 26a that is controlled to open to allow PD fluid pump 24 to pump fresh PD fluid under positive pressure through a fresh PD fluid lumen 52 of dual lumen patient line 50 to patient P. The valves also include a used PD fluid valve 26b that is controlled to open to allow PD fluid pump 24 to pull used PD fluid from patient P under negative pressure through a used PD fluid lumen 54 of dual lumen patient line 50. The valves also include one or more supply valve 26m that is controlled to open to allow fresh PD fluid to be pulled from one or more fresh PD fluid source. The valves further include a drain valve 26n that is controlled to allow used PD fluid to be delivered to a house drain or drain container via a drain line 60.

Machine or cycler 20 in the illustrated embodiment also includes pressure sensors, such as pressure sensors 28a. 28b. Pressure sensor 28a is located just downstream from fresh PD fluid valve 26a, while pressure sensor 28b is located just upstream from used PD fluid valve 26. Pressure sensor 28a may accordingly sense the pressure in fresh PD fluid lumen 52 of dual lumen patient line 50 even if fresh PD fluid valve 26a is closed, while pressure sensor 28b may sense the pressure in used PD fluid lumen 54 of dual lumen patient line 50 even if used PD fluid valve 26b is closed. Additionally, pressure sensor 28a is positioned to sense the pressure of fresh PD fluid upstream from the filter membranes discussed herein during a patient fill. Pressure sensor 28b perhaps more importantly is positioned to sense the pressure of fresh PD fluid downstream from the filter membranes discussed herein during a patient fill.

Pump 24 and valves 26a to 26n in the illustrated embodiment are under the automatic control of a control unit 40 provided by machine or cycler 20 of system 10, while pressure sensors 28a, 28b (and other sensors) output to control unit 40. Control unit 40 in the illustrated embodiment includes one or more processor 42, one or more memory 44 and a video controller 46. Control unit 40 receives, stores and processes signals or outputs from pressure sensors 28a, 28b, and other sensors provided by machine or cycler 20, such as one or more temperature sensor 30 and one or more conductivity sensor (not illustrated). Control unit 40 may use pressure feedback from one or more of pressure sensor 28a, 28b to control PD fluid pump 24 to pump dialysis fluid at a desired pressure or within a safe pressure limit (e.g., within 0.21 bar (three psig) of positive pressure to a patient's peritoneal cavity and −0.10 bar (−1.5 psig) of negative pressure from the patient's peritoneal cavity).

Control unit 40 uses temperature feedback from one or more temperature sensor 30 for example to control a heater 32, such as an inline heater to heat fresh PD fluid to a desired temperature, e.g., body temperature or 37° C. In one embodiment, heater 32 is used additionally to heat a disinfection fluid, such as fresh PD fluid, to disinfect PD fluid pump 24, valves 26a to 26n, heater 32 and all reusable fluid lines within machine or cycler 20 to ready the machine or cycler for a next treatment. The additional filtration discussed herein provides a layer of protection in addition to the heated fluid disinfection to ensure that the PD fluid is safe for delivery to patient P.

Video controller 46 of control unit 40 interfaces with a user interface 48 of machine or cycler 20, which may include a display screen operating with a touchscreen and/or one or more electromechanical button, such as a membrane switch. User interface 48 may also include one or more speaker for outputting alarms, alerts and/or voice guidance commands. User interface 48 may be provided with the machine or cycler 20 as illustrated in FIG. 1 and/or be a remote user interface operating with control unit 40. Control unit 40 may also include a transceiver (not illustrated) and a wired or wireless connection to a network, e.g., the internet, for sending treatment data to and receiving prescription instructions from a doctor's or clinician's server interfacing with a doctor's or clinician's computer.

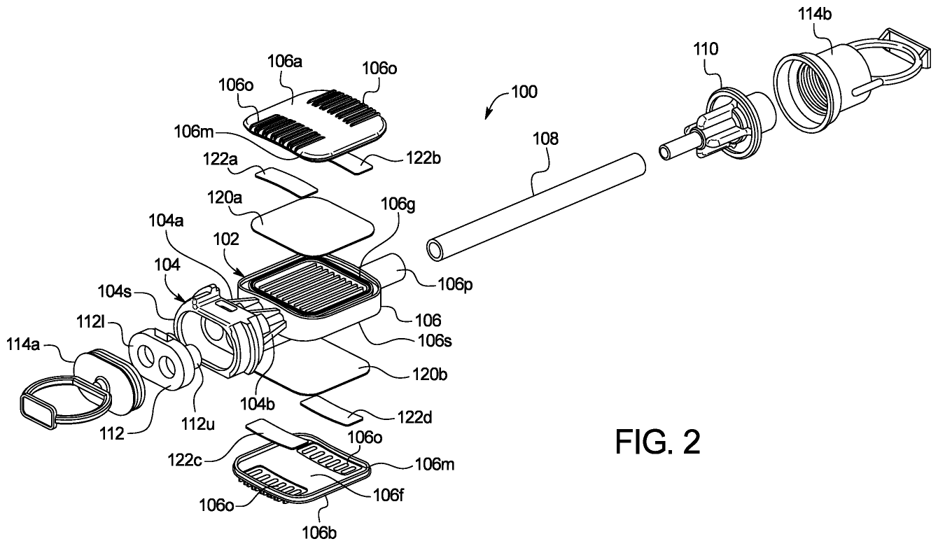

Referring to FIGS. 1 and 2, as mentioned above, fresh and used PD fluid lumens 52 and 54 of dual lumen patient line 50 may again be reusable or disposable. In the instance in which dual lumen patient line 50 is reusable, the lumens terminate with a connector 56 that connects to a lumen-side connector 104 of filter set 100, which may be sealed to (e.g., ultrasonically sealed, heat sealed, solvent bonded, laser welded or sealed via ultraviolet (“UV”) cured adhesive) or molded with a body 106 of the filter set. Body 106 in the illustrated embodiment is connected to a short, e.g., flexible, tube 108 that extends to a transfer set-side connector 110, which connects directly to a mating connector of the patient's transfer set 58. Short, e.g., flexible, tube 108 allows rigid lumen-side connector 104 and body 106 to be separated from rigid transfer set-side connector 110 to aid patient comfort. Forming body 106 to include transfer set-side connector 110, or attaching transfer set-side connector 110 to body 106, and then connecting those rigid structures to the patient's rigid transfer set 58 may lead to a combined rigid assembly that is uncomfortably tethered to patient P. The space provided by tube 108 separates body 106 from transfer set-side connector 110 so that only the rigid transfer set-side connector is mechanically connected to the patient's transfer set 58. In an alternative embodiment, however, transfer-side connector 110 may be formed with or attached to body 106.

If dual lumen patient line 50 is disposable, lumen-side connector 104 may alternatively simply include ports, e.g., fresh and used PD fluid ports 104f and 104u, to which fresh and used PD fluid lumens 52 and 54 respectively extend over or into for ultrasonic sealing, heat sealing, solvent bonding, laser welding or sealing via UV cured adhesive to the ports. In the illustrated embodiment, fresh and used ports 104f and 104u are surrounded by a threaded shroud 104s, which may make a luer type connection with mating patient line connector 56. A compressible gasket 112 is provided to seal ports 104f and 104u to mating ports of patient line connector 56 as discussed in detail herein. Caps 114a and 114b are provided (assuming dual lumen patient line 50 is reusable) on either end of filter set 100 after the set is sterilized, e.g., via irradiation, steam or ethylene oxide, to maintain sterility. To use filter set 100, the patient or user removes and discards caps 114a and 114b.

Lumen-side connector 104 and body 106 may be referred to herein as a filter housing 102. Filter housing 102, transfer set-side connector 110, caps 114a and 114b, and any other rigid or semi-rigid polymer associated with filter set 100 may be made of any one or more plastic, such as, polystyrene (“PS”), polycarbonate (“PC”), blends of polycarbonate and acrylonitrile-butadiene-styrene (“PC/ABS”), polyvinyl chloride (“PVC”), polyethylene (“PE”), polypropylene (“PP”), polyesters like polyethylene terephthalate (“PET”) or a polyester elastomer, or polyurethane (“PU”). Compressible gasket 112 may be formed from silicone rubber, PVC or other similar elastomeric material, such as styrene-ethylene-butylene-styrene (“SEBS”) or isoprene. Flexible tube 108 may be made of plasticized PVC or a non-PVC material, such as poly butadiene (“PBD”) or PP.

FIG. 2 also illustrates that lids 106a, 106b are ultrasonically sealed, heat sealed, solvent bonded or laser welded or sealed via UV cured adhesive to the sidewalls 106s of body 106 to complete the body. Prior to sealing lids 106a, 106b to the sidewalls 106s of body 106, first and second flat sheet filter membranes 120a, 120b, are ultrasonically sealed, heat sealed, solvent bonded, laser welded or sealed via UV cured adhesive at their perimeters to inner portions of the sidewalls 106s of body 106. First and second flat sheet filter membranes 120a, 120b are made in one embodiment of a hydrophilic material that may have a pore size of about 0.2 micron through which fresh PD fluid flows for further filtration. Filter membranes 120a, 120b may be made of, for example, polysulfone or polyethersulfone blended with polyvinylpyrrolidone. In an embodiment, the size and resulting surface area of filter membranes 120a, 120b may be based at least in part on one or more of the expected operating PD fluid pressures and flowrates provided by PD machine or cycler 20.

FIG. 2 further illustrates that lids 106a, 106b may be provided with vent openings 1060 that allow air to be vented from the fresh PD fluid prior to being filtered through filter membranes 120a, 120b. To maintain sterility within body 106, hydrophobic membranes 122a. 122b, 122c, 122d, etc., are ultrasonically sealed, heat sealed, solvent bonded, laser welded or sealed via UV cured adhesive at their perimeters to the inside surfaces of their respective lids 106a, 106b and so as to surround their respective vent openings 1060. Hydrophobic membranes 122a, 122b, 122c, 122d, etc., may be made for example from polytetrafluoroethylene (“PTFE”). While multiple sets of vent openings 1060 and corresponding hydrophobic membranes 122a, 122b, 122c, 122d are shown as being provided with each lid 106a, 106b, (i) only a single set of vent openings and corresponding hydrophobic membrane may be provided with each lid 106a, 106b or (ii) one or more set of vent openings and corresponding hydrophobic membrane may be provided with a single lid 106a, 106b only.

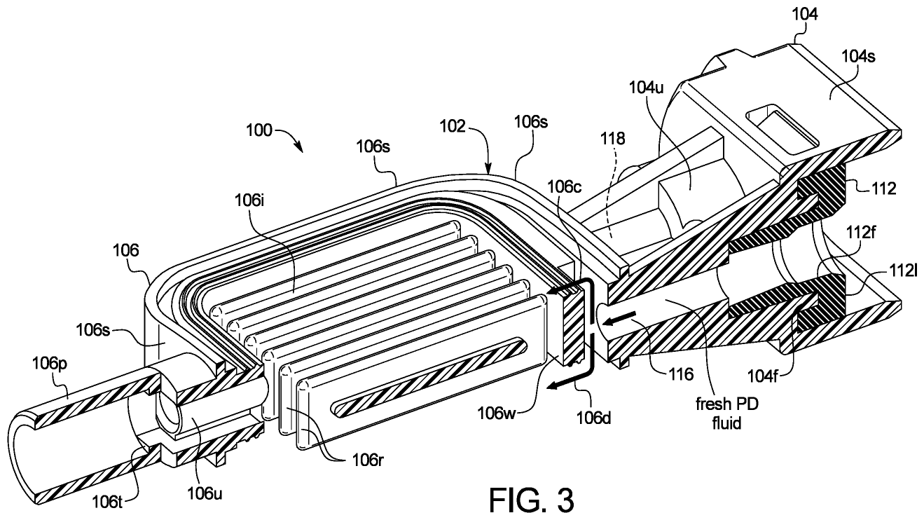

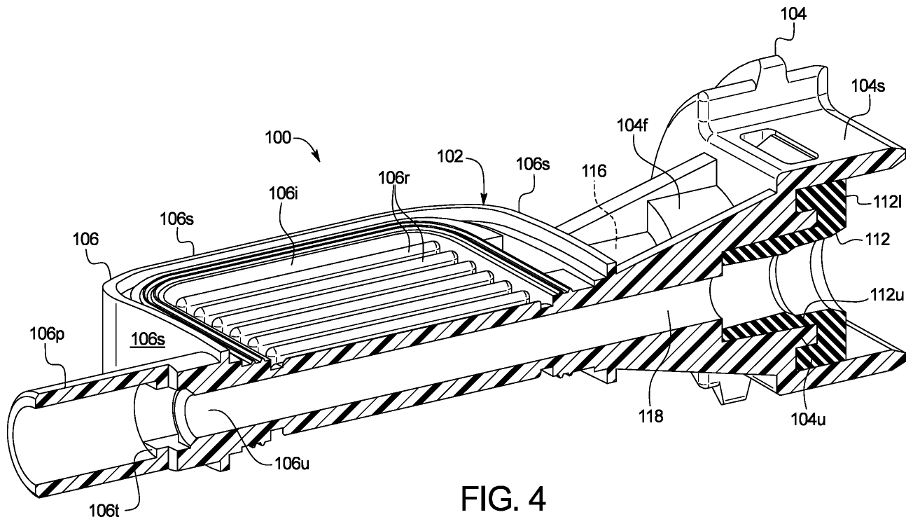

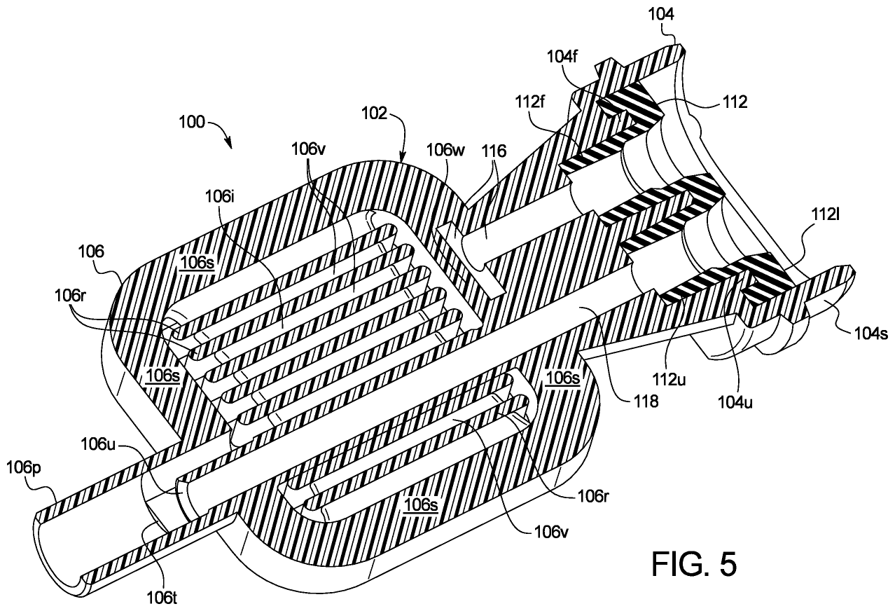

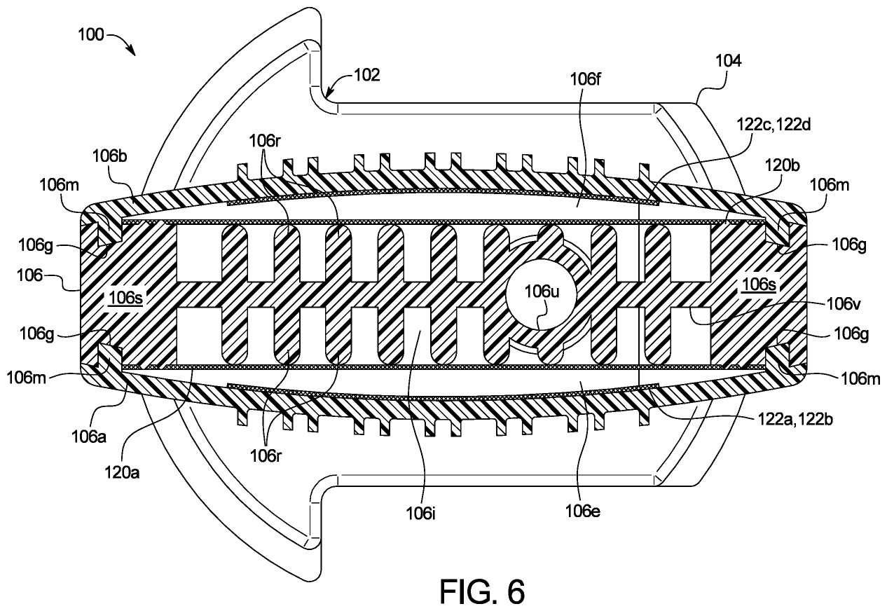

Referring not to FIGS. 3 to 6, lumen-side connector 104 and body 106 of filter housing 102 and gasket 112, which may be molded as a single structure except for lids 106a. 106b, are illustrated in more detail. Lumen-side connector 104 and body 106 form a fresh PD fluid passageway 116 and a used PD fluid passageway 118. As illustrated in FIG. 3, fresh PD fluid passageway 116 extends through fresh PD fluid port 104f in lumen-side connector 104 and towards a deflecting wall 106w located within body 106 of filter housing 102. Deflecting wall 106w forces the fresh PD fluid to split outwardly in two directions forming a first outer compartment fresh PD fluid pathway 106c and a second outer compartment fresh PD fluid pathway 106d. First outer compartment fresh PD fluid pathway 106c extends in a first direction along and then past deflecting wall 106w and makes a turn, such as a right angle turn, into a first outer compartment 106e (FIG. 6), which resides over the outside of first flat sheet filter membrane 120a. Second outer compartment fresh PD fluid pathway 106d extends in a second direction along and then past deflecting wall 106w and likewise makes a turn, such as a right angle turn, into a second outer compartment 106f (FIG. 6), which resides over the outside of a second flat sheet filter membrane 120b. Outer compartments 106e, 106f are sized to displace the fresh PD fluid across an upstream sides of filter membranes 120a. 120b for even distribution through the membranes. Fresh PD fluid is pressurized within the first and second outer compartments 106e. 106f. The pressurization forces the fresh PD fluid through filter membranes 120a. 120b and into an inner compartment 106i of body 106, which is bounded in part by the inner surfaces of the first and second flat sheet filter membrane 120a. 120b.

Filter membranes 120a. 120b may be bacteria reduction or sterilizing grade hydrophilic membranes, which may be formed with porous walls having a pore size of about 0.2 micron through which the fresh PD fluid flows for further filtration. Providing multiple flat sheet membranes 120a. 120b enables the membranes and thus filter housing 102 of the filter set to be shorter, while providing the necessary filtration surface area needed over multiple patient fills of a PD treatment. A shorter filter housing 102 is better for patient comfort because patient P (FIG. 1) is typically sleeping near filter set 100 during treatment. It should be appreciated however that a single filter membrane 120a or 120b may be provided alternatively.

Fresh and further filtered PD fluid flows in one embodiment from the inner compartment 106i of body 106, through a transfer set-side port 106p of the body, through short, e.g., flexible, tube 108 of filter set 100, and through transfer set 58, into the patient P's peritoneal cavity. As illustrated in FIGS. 3 to 5, transfer set-side port 106p in one embodiment includes an internal stop 106t that sets an inserted location for tube 108, or at least provides a location past which short tube 108 can no longer be inserted into transfer set-side port 106p. Internal stop 106t is in one embodiment set off from the beginning or end of an internal used PD fluid tube 106u, which may be molded with the main portion of body 106. Used PD fluid tube 106u extends through inner compartment 106i of body 106 and partially into used PD fluid port 104u provided by lumen-side connector 104. Used PD fluid port 104u is in sealed fluid communication during operation to used PD fluid lumen 54 of dual lumen patient line 50.

Used PD fluid tube 106u enables used PD fluid to be pulled through body 106 of filter housing 102 without contacting and potentially clogging either of the filter membranes 120a. 120b. Used PD fluid tube 106u also provides a clear, straight path for the used PD fluid (e.g., having an inner diameter of 2 millimeters (“mm”) to 8 mm, such as about 3 mm or 4 mm), which helps to mitigate against pressure losses due to filter set 100. While it is fluidically possible for used PD fluid to flow along the outside of used PD fluid tube 106u into inner compartment 106i of body 106, negative pressure is applied only from within used PD fluid tube 106u, so there is little incentive for used PD fluid to flow along the outside of the used PD fluid tube. Likewise, while it is fluidically possible for fresh PD fluid to flow in reverse up used PD fluid tube 106u, the change in direction required makes such a path much more tortuous than simply flowing through transfer set-side port 106p to patient P. Also, used PD fluid tube 106u and used PD fluid lumen 54 of dual lumen patient tube 50 are likely full of PD fluid during a patient fill, and the used PD fluid lumen is closed off at PD machine or cycler 20, so there is little or no room for fresh PD fluid to enter the used PD fluid tube.

Inner compartment 106i of body 106 is provided in one embodiment with a series of ribs 106r that support first and second flat sheet filter membranes 120a, 120b (FIG. 6) when placed under negative fluid pressure. In this manner, filter membranes 120a, 120b and inner compartment 106i may be as large as they need to be to provide a desired filtration capacity. The series of ribs 106r and their supporting structure 106v are co-molded with used PD fluid tube 106u and sidewalls 106s of body 106 in one embodiment. FIG. 6 illustrates that first and second flat sheet filter membranes 120a, 20b are sealed in place to inner portions of sidewalls 106s of body 106 via ultrasonic sealing, heat sealing, solvent bonding, laser welding or sealing via UV cured adhesive.

FIGS. 2 and 6 illustrate that body 106 of filter housing 102 includes first and second lids 106a, 106b, which may be formed from the same material as the remainder of the body. First and second lids 106a, 106b may be ultrasonically sealed, heat sealed, solvent bonded, laser welded or sealed via UV cured adhesive to outer portions of sidewalls 106s of body 106. A tongue and groove fit, e.g., via tongues 106m formed with lids 106a, 106b and grooves 106g formed in sidewalls 106s, may be provided between first and second lids 106a, 106b and sidewalls 106s to center the lids for sealing and/or to serve as a flash trap for material melted by the weld or other process used to seal the lids to the body. First and second lids 106a, 106b respectively form the outsides of first and second outer compartments 106e, 106f into which fresh PD fluid flows before passing through filter membranes 120a, 120b.

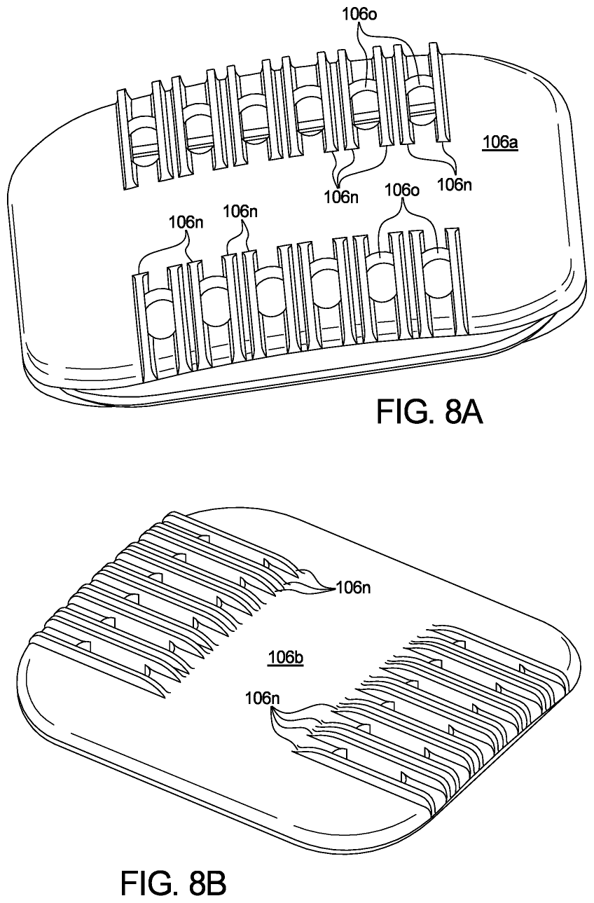

First and second lids 106a, 106b may be formed with one or more air vent 1060 (FIGS. 2, 8A, 8B). FIGS. 2 and 6 show that each vent 1060 is covered on the inside of the respective lid 106a, 106b with a hydrophobic membrane 122a, 122b, 122c, 122d, which may be ultrasonically sealed, heat sealed, solvent bonded, laser welded or sealed via UV cured adhesive to the inside of the respective lid. One or more hydrophobic membrane 122a, 122b, 122c, 122d allows air to be vented to atmosphere during priming and at anytime during treatment when the fresh PD fluid is pressurized within outer compartments 106e, 106f of body 106 prior to being filtered through hydrophilic sheet membranes 120a, 120b, which may improve the performance of the membranes in addition to removing air from filter set 100.

Regarding the priming of filter set 100 for treatment, fresh PD fluid lumen 52 of patient line 50 and filter set 100 may or may not be primed with fresh PD fluid before short tube 108 is connected to the patient's transfer set 58. If primed, user interface 48 may audibly, visually or audiovisually prompt patient P to clip patient line connector 56 and/or filter set 100 into a clip provided by housing 22 of PD machine or cycler 20. As illustrated in FIG. 2, short tube 108 is initially fitted with a cap 114b, so that when patient line connector 56 or filter set 100 is clipped to housing 22, short tube 108 hangs off of filter set 100 and is closed to the environment via cap 114b. Control unit 40 then causes PD fluid pump 24, with fresh PD fluid valve 26a open and used PD fluid valve 26b closed, to prime fresh PD fluid lumen 52 with fresh PD fluid up to filter membranes 120a, 120b. Here, air is forced out vent openings 1060.

Once fresh PD fluid lumen 52 is primed fully, pressure sensors 28a and 28b sense a pressure increase because fresh PD fluid has nowhere to go with used PD fluid valve 26b closed. Upon seeing the pressure increase, with filter membranes 120a, 120b now fully wetted, control unit 40 then causes used PD fluid valve 26b to open, allowing PD fluid pump 24 to push fresh PD fluid through hydrophilic filter membranes 120a. 120b into inner compartment 106i, which pushes air through the inner compartment, into and through used PD fluid tube 106u, and into a portion of used PD fluid lumen 54. Air is accordingly pushed up the used PD fluid lumen 54 towards system drain. Here, control unit 40 may be programed to know and actuate a number of known volume strokes of PD fluid pump 24 needed to adequately prime inner compartment 106i, used PD fluid tube 106u, and a desired portion of used PD fluid lumen 54. At this point, body 106 of filter set 100 is fully primed. It should be appreciated that filter set 100 does not have to be clamped to housing 22 for the above priming of the body 106 of filter set 100 to be performed, however, doing so may help to prevent dual lumen patient line 50 from kinking during such priming.

User interface 48 of PD machine or cycler 20 then audibly, visually or audiovisually prompts patient P to remove filter set 100 from the clip at housing 22, to remove cap 114b from short tube 108, to connect short tube 108 to the patient's transfer set 58, and to open the clamp of the patient's transfer set 58. Control unit 40 then in an embodiment, with used PD fluid valve 26b open and fresh PD fluid valve 26a open or closed (likely closed), causes PD fluid pump 24 to pull used PD fluid from the patient to prime short tube 108, here pulling air from the short tube, through used PD fluid tube 106u, up used PD fluid lumen 54 of dual lumen patient line 50, and towards the drain of PD machine or cycler 20. Such pulling of used PD fluid may be part of an initial drain of the patient. The amount of used PD fluid removed from the patient is accordingly counted at control unit 40 (e.g., by accumulating known volume strokes of PD fluid pump 24) as part of the treatment's initial drain volume in one embodiment.

If a patient fill is instead the first action to be taken after priming fresh PD fluid lumen 52 and the body 106 of filter set 100, control unit 40 may or may not pull effluent from the patient to fully prime short tube 108 prior to starting the initial patient fill. That is, it is contemplated to allow the small amount of air residing within short tube 108 to be pushed back to the patient. If however, control unit 40 does pull an initial amount of effluent from the patient to prime short tube 108, control unit 40 may count whatever amount of effluent is pulled from the patient (e.g., by accumulating known volume strokes of PD fluid pump 24) as part of a subsequent initial drain.

In an alternative embodiment, filter set 100 is not clipped at housing 22 and short tube 108 is initially connected to the patient's transfer set 58. User interface 48 here audibly, visually or audiovisually counsels patient P to leave the clamp of the patient's transfer set 58 closed until instructed to open the clamp. The procedure described above is then performed, wherein here the patient's transfer set clamp is performing the function of cap 114b at the end of short tube 108 in the above example. With the patient's transfer set clamp closed, control unit 40 causes fresh PD fluid to be primed through fresh PD fluid lumen 52, body 106 of filter set, and a portion of used PD fluid lumen 54 using PD fluid pump 24, while sequencing valves 26a and 26b as discussed above.

User interface 48 then prompts patient P to open the clamp of the patient's transfer set 58 and to press a confirm button at user interface 48 in one embodiment. Upon the confirm button being pressed, control unit 40 then sequences valves 26a and 26b and actuates pump 24 as discussed above to pull used PD fluid from the peritoneal cavity of patient P to prime short tube 108 and used PD fluid tube 106u with patient effluent. The effluent priming of short tube 108 may again be part of an initial patient drain.

The pulling of used PD fluid from the patient to prime short tube 108 assumes that there is used PD fluid to remove from the patient at the start of treatment. This is true in many instances in which the patient is full of used PD fluid at the beginning of treatment from a previous treatment's last fill or from a midday exchange. In some instances, however, the patient is dry at the beginning of treatment. It is contemplated that control unit 40 of PD machine or cycler 20, which may be dedicated at a given time to a single patient, knows the patient's treatment schedule, and thus knows when the patient will begin a next treatment in a dry state with no or very little used PD fluid. It is contemplated here that control unit 40, instead of attempting to completely drain the patient in a final drain of a previous treatment, causes a residual amount of effluent to remain within the peritoneal cavity of the patient after treatment. The residual amount may for example be 50 milliliters (“ml”) or more as needed to ensure that the patient's indwelling PD catheter can access the residual effluent. The residual amount should be enough to prime any air at least through the proximal end of short tube 108 at the junction of filter set 100.

The above-described priming procedure is advantageous for a number of reasons. First, a step of having the patient clip patient line connector 56 into a clip provided by housing 22 of PD machine or cycler 20 may be eliminated. Also, the need for patient line connector 56 to be fitted with a vented cap and/or for housing 22 of PD machine or cycler 20 to have a sensor for detecting when fresh PD fluid has reached patient line connector 56 may be eliminated. Both savings reduce cost and complexity. Second, after treatment, the patient disconnects transfer set-side connector 110 from the patient's transfer set 58 and then seals transfer set 58 with a cap (not illustrated) having a disinfectant, such as iodine, to help prevent peritonitis due for example to patient touch contamination. The cap is then removed and replaced with a new transfer set-side connector 110 of a new filter set 100 at the beginning of a next treatment. Residual disinfectant, e.g., residual iodine, remains however. The priming method disclosed herein carries the residual disinfectant away into used PD fluid lumen 54 of dual lumen patient line 50 under negative pressure instead of delivering the residual disinfectant to the patient. Doing so may prevent health issues, especially for sensitive patients.

Turning now to FIGS. 2 to 5, gasket 112, such as a silicone or polyvinyl chloride (“PVC”) rubber, is fitted onto the fresh and used PD fluid ports 104f, 104u of lumen-side connector 104 of filter housing 102. Gasket 112 in one embodiment provides a double seal against the mating patient line connector 56. Patient line connector 56 includes fresh and used ports (not illustrated) that extend respectively into fresh and used PD fluid ports 104f, 104u of lumen-side connector 104. Gasket 112 provides fresh and used port seals 112f, 112u between the mated fresh and used PD fluid ports of patient line connector 56 and fresh and used ports 104f, 104u of lumen-side connector 104. Port seals 112f, 112u and the fresh and used ports of patient line connector 56 may be tapered so as to narrow while extending into filter housing 102. Port seals 112f, 112u provide first seals with patient line connector 56. Patient line connector 56 also includes a flange having a raised continuous rib that extends around the flange (not illustrated). The flange and the rib are translated towards a flange portion 1121 of gasket 112 when patient line connector 56 is connected to lumen-side connector 104. When patient line connector 56 is fully connected to lumen-side connector 104, the raised rib extends into the deformable flange portion 1121 of gasket 112, which compresses around the raised rib to form a second seal between lumen-side connector 104 and patient line connector 56.

Used PD fluid removed through the patient's transfer set 58 travels under negative pressure through filter set 100 via the used PD fluid tube 106u (thus bypassing filter membranes 120a, 120b), through used PD fluid lumen 54 of dual lumen patient line 50, and back to machine or cycler 20. Machine or cycler 20 pumps the used PD fluid under positive pressure to drain via drain line 60. Machine or cycler 20 includes pressure sensor 28b located along the used PD fluid side of its internal tubing, which measures the negative pressure applied by PD fluid pump 24 to the used PD fluid during a patient drain. That same pressure sensor 28b may be used during a patient fill to measure the positive pumping pressure (which is transmitted back through used PD fluid tube 106u of filter set 102 and used PD fluid lumen 54 of patient line 50 to pressure sensor 28b), which is desirable because the measured pressure is of the fresh PD fluid downstream (after filtration) of filter membranes 120a, 120b. The measured pressure accordingly takes into account any pressure drop across filter membranes 120a. 120b, which more accurately reflects the pressure at which the PD fluid is being delivered to patient P.

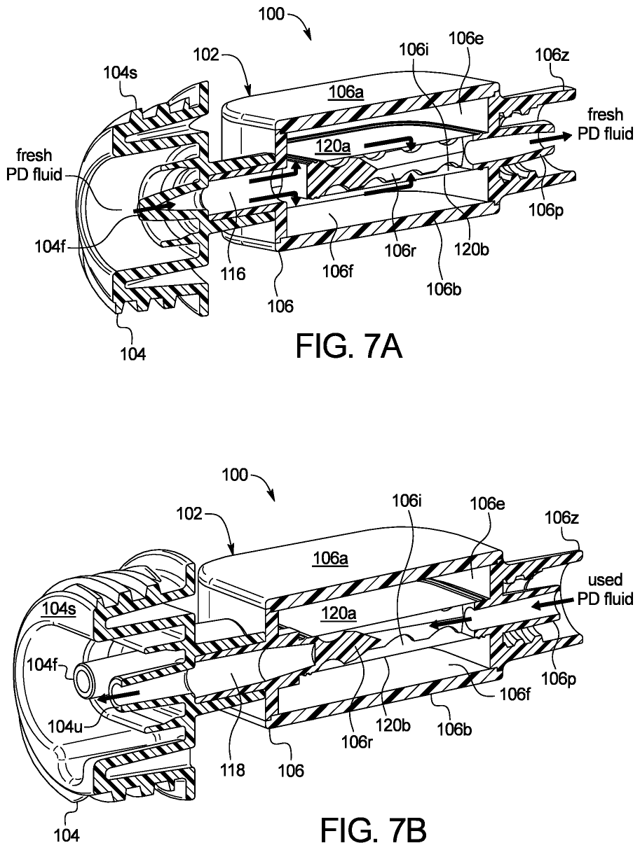

Referring now to FIGS. 7A and 7B, an alternative embodiment for a filter set 100 is illustrated. Filter set 100 in FIGS. 7A and 7B includes many of the same features as filter set 100 of FIGS. 2 to 6, which are numbered the same and include all structure, functionality and alternatives described above for same. Filter set 100 in FIGS. 7A and 7B includes lumen-side connector 104 having fresh and used ports 104f and 104u surrounded by a threaded shroud 104s. Lumen-side connector 104 is connected to or molded with body 106, which is capped via lids 106a, 106b that form first and second outer compartments 106e, 106f, respectively, along with the outside surfaces of flat sheet hydrophilic filter membranes 120a, 120b. FIG. 7A shows that fresh PD fluid is delivered via fresh PD fluid passageway 116 and pressurized within first and second outer compartments 106e. 106f so as to be further filtered through filter membranes 120a, 120b. Further filtered fresh PD fluid flows into inner compartment 106i of body 106 and from inner compartment 106i, through transfer set-side port 106p, to the patient. One difference with filter set 100 in FIGS. 7A and 7B is that transfer set-side port 106p is surrounded by a threaded shroud 1062, which mates with a mating connector for short tube 108 or for the patient's transfer set 58.

FIG. 7B illustrates a primary difference with filter set 100 in FIGS. 7A and 7B, namely, that used PD fluid tube 106u is not provided and that used PD fluid flows instead from transfer set-side port 106p, within inner compartment 106i between and around the series of ribs 106r, and out used PD fluid passageway 118 and used PD fluid port 104u of lumen-side connector 104 to used PD fluid lumen 54 of patient line 50. Used PD fluid may tangentially contact the inside surfaces of filter membranes 120a, 120b. The primary mechanism preventing used PD fluid from entering fresh PD fluid passageway 116 (during a patient drain) and fresh PD fluid from entering used PD fluid passageway 118 (during a patient fill) is the fact that (i) fresh PD fluid lumen 52 is closed or occluded at machine or cycler 20 during a patient drain and (ii) used PD fluid lumen 54 is closed or occluded at machine or cycler 20 during a patient fill. Thus even if used PD fluid does attempt to flow through membrane filters 120a, 120b and fresh PD fluid passageway 116 during a patient drain, there is no place for the used PD fluid to go as the pressure in fresh PD fluid lumen 52 is equalized. And, even if fresh PD fluid does make the turn to flow back through used PD fluid passageway 118 during a patient fill, there is no place for the fresh PD fluid to go as the pressure in used PD fluid lumen 54 is equalized.

FIGS. 8A and 8B illustrate suitable lids 106a, 106b for ultrasonically sealing, heat sealing, solvent bonding, laser welding or sealing via UV cured adhesive to body 106 for filter set 100 of either FIGS. 2 to 6 or FIGS. 7A, 7B. Here, to prevent accidental blocking of vent openings 1060 provided by lids 106a. 106b, protective features, such ribs 106n or other raised structures are provided along one or both sides of vent openings 1060, so that air can be vented through vent openings 1060 from the interior of body 106, e.g., through hydrophobic membranes 122a, 122b, 122c, 122d, even if the patient is lying on filter set 100. Protective ribs 106n to this end form an air path between lids 106a, 106b and whatever outside surface the lids are contacting when the patient is lying on filter set 100.

It should be understood that various changes and modifications to the presently preferred embodiments described herein will be apparent to those skilled in the art. It is therefore intended that any or all of such changes and modifications may be covered by the appended claims. For example, dual lumen patient line 50 could alternatively be a single lumen patient line, wherein filter set 100 includes check valves for directing fresh and used PD fluid to desired locations within the set. Also, while hydrophilic filter membranes 120a, 120b are described in one embodiment as having a pore size of about 0.2 micron, one or both of filter membranes 120a, 120b alternatively or additionally includes a charged membrane for endotoxin reduction. Further, while two filter membranes 120a, 120b are illustrated, system 10 may alternatively employ a single filter membrane or three or more filter membranes 120a to 120n.

The invention is claimed as follows:

1. A peritoneal dialysis (“PD”) system (10) comprising:
a PD machine (20);
a patient line (50) extending from the PD machine (20); and
a filter set (100) in fluid communication with the patient line (50), the filter set (100) including at least one filter membrane (120a, 120b) positioned and arranged such that fresh PD fluid flows through the at least one filter membrane (120a, 120b) prior to exiting the filter set (100), the filter set (100) further including a used PD fluid tube (106u) positioned and arranged to carry used PD fluid past the at least one filter membrane (120a, 120b) without contacting the at least one filter membrane (120a, 120b).

2. The PD system (10) of claim 1, wherein the patient line (50) is a dual lumen patient line including a fresh PD fluid lumen (52) and a used PD fluid lumen (54), the used PD fluid lumen (54) placed in fluid communication with the used PD fluid tube (106u).

3. The PD system (10) of claim 2, wherein the filter set (100) includes a fresh PD fluid port (104f) for fluid communication with the fresh PD fluid lumen (52) and a used PD fluid port (104u) for fluid communication with the used PD fluid lumen (54).

4. The PD system (10) of claim 3, wherein the used PD fluid tube (106u) is in fluid communication with the used PD fluid port (104u).

5. The PD system (10) according to claim 3, wherein the filter set (100) includes a compressible gasket configured to seal the fresh and used PD fluid ports (104f, 104u) to a patient line connector (56) located at an end of the dual lumen patient line (50).

6. The PD system (10) of claim 1, wherein the filter set (100) includes first and second filter membranes (120a, 120b) separated by an inner compartment (106i) into which fresh PD fluid is filtered by the first and second filter membranes (120a, 120b), and wherein the used PD fluid tube (106u) extends through the inner compartment (106i).

7. The PD system (10) of claim 6, which includes at least one rib (106r) located within the inner compartment (106i) for supporting the first and second filter membranes (120a, 120b).

8. The PD system (10) of claim 1, wherein the filter set (100) includes a transfer set-side port (106p) within which fresh and used PD fluid flows, the used PD fluid tube (106u) extending into the transfer set-side port (106p).

9. The PD system (10) of claim 8, wherein the transfer set-side port (106p) defines an internal stop (106t) against which a tube (108) may be abutted, the internal stop (106t) spaced from an end of the used PD fluid tube (106u) extending into the transfer set-side port (106p).

10. The PD system (10) of claim 1, wherein the filter set (100) includes at least one outer compartment (106e, 106f) sized to displace the fresh PD fluid across an upstream side of the at least one filter membrane (120a, 120b).

11. The PD system (10) of claim 10, wherein the at least one filter membrane (120a, 120b) is a flat sheet filter membrane.

12. The PD system (10) of claim 10, wherein the filter set (100) includes a deflecting wall (106w) positioned and arranged to displace incoming fresh PD fluid towards the at least one outer compartment (106e, 106f).

13. The PD system (10) of claim 10, wherein the filter set (100) includes at least one lid (106a, 106b) cooperating with the at least one filter membrane (120a, 120b) to form the at least one outer compartment (106e, 106f).

14. The PD system (10) of claim 13, wherein the at least one lid (106a, 106b) includes at least one vent opening (1060) and at least one hydrophobic membrane (122a, 122b, 122c, 122d) sealingly covering the at least one vent opening (1060).

15. The PD system (10) of claim 13, wherein the filter set (100) includes at least one sidewall (106s), wherein the at least one lid (106a, 106b) is sealed to an outer portion of the at least one sidewall (106s), and wherein the least one filter membrane (120a, 120b) is sealed to an inner portion of the at least one sidewall (106s).

16. The PD system (10) of claim 1, wherein the filter set (100) is configured to connect directly to a patient's transfer set, or wherein the filter set (100) includes a flexible tube (108) configured to connect to the patient's transfer set.

17. The PD system (10) of claim 1, wherein the PD machine (20) includes a pressure sensor (28b) positioned and arranged to sense the pressure of fresh PD fluid downstream from the at least one filter membrane (120a, 120b) during a patient fill.

18. The PD system (10) of claim 1, wherein the at least one filter membrane (120a, 120b) is a bacteria reduction filter membrane or a sterilizing grade filter membrane.

19. A peritoneal dialysis (“PD”) system (10) comprising:
a PD machine (20);
a patient line (50) extending from the PD machine (20); and
a filter set (100) in fluid communication with the patient line (50), the filter set (100) including first and second filter membranes (120a, 120b) separated by an inner compartment (106i) into which fresh PD fluid is filtered by the first and second filter membranes (120a, 120b), and wherein the filter set (100) is further configured such that used PD fluid flows through the inner compartment (106i).

20. The PD system (10) of claim 19, which includes at least one rib (106r) located within the inner compartment (106i) for supporting the first and second filter membranes (120a, 120b), the used PD fluid flowing around the at least one rib (106r) before exiting the inner compartment (106i).

21. A filter set (100) comprising:
a body (106);
at least one filter membrane (120a, 120b) positioned and arranged within the body (106) such that fresh peritoneal dialysis (“PD”) fluid flows through the at least one filter membrane (120a, 120b) prior to exiting the filter set (100); and
a used PD fluid tube (106u) positioned and arranged within the body (106) to carry used PD fluid past the at least one filter membrane (120a, 120b) without contacting the at least one filter membrane (120a, 120b).

22. A filter set (100) comprising:
a body (106);
at least one filter membrane (120a, 120b) positioned and arranged within the body (106) such that fresh peritoneal dialysis (“PD”) fluid flows through the at least one filter membrane (120a, 120b) prior to exiting the filter set (100); and
at least one lid (106a, 106b) including at least one vent opening (1060) and at least one protective rib (106n) located adjacent to the at least one vent opening (1060) to maintain the at least one vent opening (1060) in an uncovered condition during operation.

23. A method for priming a filter set (100) connected to a dual lumen patient line (50), wherein during treatment a tube (108) is located between the filter set (100) and a patient's transfer set (58), the method comprising:
delivering fresh peritoneal dialysis (“PD”) fluid through a fresh PD fluid lumen (52) of the dual lumen patient line (50) to the filter set (100);
forcing the fresh PD fluid through at least one filter membrane (120a, 120b) of the filter set (100), so that the fresh PD fluid displaces air towards a used PD fluid lumen (54) of the dual lumen patient line (50); and
pulling used PD fluid from the patient, through the patient's transfer set (58), through the tube (108), through a used PD fluid portion of the filter set (100), and into the used PD fluid lumen (54) of the dual lumen patient line (50).

24. The method of claim 23, wherein pulling used PD fluid is provided as part of an initial patient drain.

25. The method of claim 23, wherein between forcing the fresh PD fluid through the at least one filter membrane (120a, 120b) and pulling used PD fluid from the patient, the patient is prompted to connect the tube to the patient's transfer set (58).

26. The method of claim 23, wherein between forcing the fresh PD fluid through the at least one filter membrane (120a, 120b) and pulling used PD fluid from the patient, the patient is prompted to open a clamp of the patient's transfer set (58).

27. The method of claim 23, wherein air is primed through at least one vent opening (1060) of the filter set (100) while delivering fresh PD fluid through the fresh PD fluid lumen (52) of the dual lumen patient line (50).

28. The method of claim 23, wherein a fresh PD fluid valve (26a) is open and a used PD fluid valve (26b) is closed while delivering fresh PD fluid through the fresh PD fluid lumen (52) of the dual lumen patient line (50).

29. The method of claim 23, wherein a used PD fluid valve (26b) is open while forcing the fresh PD fluid through the at least one filter membrane (120a, 120b) of the filter set (100).

30. The method of claim 23, wherein a used PD fluid valve (26b) is open while pulling used PD fluid from the patient.

31. The method of claim 23, which includes accumulating known volume pump strokes to control a volume pumped to force the fresh PD fluid through the at least one filter membrane (120a, 120b).

32. The method of claim 23, which includes sensing a pressure increase to transition from (i) delivering fresh PD fluid through the fresh PD fluid lumen (52) of the dual lumen patient line (50) to the filter set (100) to (ii) forcing the fresh PD fluid through the at least one filter membrane (120a, 120b).

33. The method of claim 23, wherein the used PD fluid portion of the filter set (100) includes a used PD fluid tube (106u).

34. The method of claim 23, wherein the used PD fluid pulled from the patient is residual effluent from a previous treatment left for the purpose of priming the tube (108).

35. The method of claim 34, wherein a volume of the residual effluent is at least 50 ml.
